(19) United States
(12) Patent Application Publication
(10) Pub. No.: US 2023/0293202 A1
(43) Pub. Date: Sep. 21, 2023

(54) IMPLANT DETACHMENT MECHANISM

(71) Applicant: SAHAJANAND MEDICAL TECHNOLOGIES LIMITED, Gujarat (IN)

(72) Inventors: Abhijeet SINGHVI, Gujarat (IN); Chirag Maheshbhai Solanki, Gujarat (IN); Shivanshu Pandey, Uttar Pradesh (IN); Akashkumar Ashokbhai Munjani, Surat (IN); Isha Vashisht, Haryana (IN)

(21) Appl. No.: 17/875,204

(22) Filed: Jul. 27, 2022

Related U.S. Application Data
(63) Continuation of application No. PCT/IN2022/050540, filed on Jun. 13, 2022.

(30) Foreign Application Priority Data
Mar. 21, 2022 (IN) 202221015511

(51) Int. Cl. A61B 17/34; A61B 90/00

(52) U.S. Cl. CPC A61B 17/3468; A61B 90/39; A61B 2017/00473

(57) ABSTRACT

An implant holder (140) is disposed in a catheter delivery system (100). The implant holder (140) engages a portion of the implant to restrain its relative movement in axial and radial direction. The implant holder includes specific provisions in form of specifically designed thin strips (206, 306, 406, 506, 606, 706, 806) to assist in detachment of the implant from the implant holder. The thin strips are designed to utilize elastic properties of the material to store and release potential energy during the procedure.

### CROSS REFERENCE(S) TO RELATED APPLICATIONS

This application is a continuation of PCT Application No. PCT/IN2022/050540, filed Jun. 13, 2022, which claims priority to Indian Application No. IN202221015511, filed Mar. 21, 2022 the entire contents of each being hereby incorporated by reference.

### TECHNICAL FIELD

The technology described herein relates to a percutaneous implant delivery system and methods to deliver an implant inside a human or animal body. Specifically, the technology is related to a detachment mechanism for assisting in deployment of a coronary or a peripheral implant.

### BACKGROUND

Healthy and proper functioning of organs e.g. heart, brain, liver, pancreas, kidneys, cardiovascular system etc., along with their internal parts e.g. for a heart its arteries, veins, valves, nodes, walls, and remaining constituents, is essential for healthy living and well-being of a human or an animal. However, due to factors like age, disease, infections or genetic disorder, the working efficiency of the organs reduce significantly and many times that results in a potentially life-threatening condition. Conventionally, surgery was one main option to operate on the severely diseased organ e.g., replacing the diseased heart valve by a mechanical heart valve or bypassing or removing a blocked artery using a harvested artery etc. However, in recent years, alternative less invasive transcatheter-based approaches have been developed where a percutaneous catheter delivers an implant transvascularly through variety of access points in a cardiovascular network e.g., through femoral artery, transapically, transaortic, trans-axillary etc. These implants may be, but not limited to, a stent, a valve, a mesh, a balloon, a patch, a drug-containing matrix, a shunt, or a combination thereof.

During a transvascular procedure, a catheter delivery system, carrying an implant, plays a vital role as the operator’s maneuvering actions at proximal end (handle) of the delivery system directly impacts the positioning, movement of the distal section (tip and capsule), and performance of the implant after the deployment. The effect of maneuvering actions transfers through a catheter shaft from the proximal end to the distal end. The catheter shaft is situated between the proximal end and the distal end. However, sometimes, the implant doesn’t get detached from the delivery system quickly and requires additional maneuvering that consumes additional time and may also reduce accuracy in positioning of the implant.

Hence, there is a need to provide a detachment mechanism in a catheter delivery system for trans-vascularly deliver an implant to avoid the shortcomings known in the art and specifically to provide a catheter delivery system that gives precision and efficiency in detachment and deployment of the implants.

### SUMMARY

The subject technology is illustrated, for example, according to various aspects described below.

In some example embodiments, an implant detaching mechanism to detach an implant from an implant holder, comprises a catheter having an inner shaft, a catheter shaft, a capsule, and a handle. The implant holder is attached to the inner shaft and having at least one pin on its peripheral surface to engage an implant to the implant holder. The catheter shaft is movable in the longitudinal direction and a capsule is attached to the distal end of the catheter shaft wherein, on movement of the catheter shaft, the capsule covers and uncovers the implant holder and at least the engaged implant partially. A thin strip, elastic in nature, is attached to the implant holder and placed between the peripheral surface of the implant holder and the engaged implant. On moving the capsule to cover the implant holder, a compressive force is being applied on the thin strip and the engaged implant. On moving the capsule to uncover the implant holder, release of potential energy stored in the thin strip causes detachment of the engaged implant from the pin.

It will be appreciated that the above-described features are merely examples and other features, aspects, and advantages of the subject matter are further illustrated in the figures and described in the corresponding description below.

### BRIEF DESCRIPTION OF ACCOMPANYING DRAWINGS

The detailed description is described with reference to the accompanying figures.

FIG. 1 illustrates an isometric view of a catheter delivery system, according to some embodiment;

FIG. 1A illustrates a detailed view of a capsule assembly showing position of an implant holder in the catheter delivery system of FIG. 1, according to certain example embodiments;

FIGS. 2 and 2A illustrate, respectively, a detailed isometric view and a detailed side view of an implant detachment mechanism in an implant holder of the catheter delivery system of FIG. 1, according to certain example embodiments;

FIG. 3 illustrates a detailed isometric view of an implant detachment mechanism in an implant holder in the catheter delivery system of FIG. 1, according to certain example embodiments;

FIG. 4 illustrates a detailed isometric view of an implant detachment mechanism in an implant holder in the catheter delivery system of FIG. 1, according to certain example embodiments;

FIGS. 5 and 5A illustrate, respectively, a detailed isometric view and a detailed side view of an implant detachment mechanism in an implant holder of the catheter delivery system FIG. 1, according to certain example embodiments;

FIGS. 6 and 6A illustrate, respectively, a detailed isometric view and a detailed side view of an implant detachment mechanism in an implant holder of the catheter delivery system of FIG. 1, according to certain example embodiments;

FIGS. 7 and 7A illustrate, respectively, a detailed isometric view and a detailed side view of an implant detachment mechanism in an implant holder of the catheter delivery system of FIG. 1, according to certain example embodiments; and

FIGS. 8 and 8A illustrate, respectively, a detailed isometric view and a detailed side view of an implant detachment mechanism in an implant holder of the catheter delivery system of FIG. 1, according to certain example embodiments.

### DETAILED DESCRIPTION

According to the present disclosure, in some embodiments, a catheter delivery system, for trans-vascularly delivering and deploying an implant in a human heart, comprises a detachment mechanism to ensure disconnect or detachment of the implant from the catheter delivery system once the implant reaches its deployment location and it is in correct positioning too. By definition, the detachment of the implant from the catheter delivery system is an incident after which the implant cannot be maneuvered anymore and the next process steps of retraction of the catheter from the deployment site initiate.

In one non-limiting aspect, a typical catheter delivery system comprises a distal section, a middle section, and a proximal section. The proximal section remains outside the human body and comprises a handle housing that encompasses mechanisms to control the movements at the distal section of the catheter. The distal section comprises a tip, an inner shaft, a guidewire shaft, an implant holder, and a capsule wherein, in a loaded state, the distal section comprises an implant too. The middle section is connected proximally with the handle housing and distally it connects to the distal section.

The capsule is a hollow, cylindrical structure that is movable through movement mechanisms present in the middle section and actuated from the proximal section. The capsule provides an inside space where the implant is loaded in compressed form and the capsule helps in retaining the implant in compressed form. Usually, capsule-based catheter delivery systems are used for delivery of implants whose frame structure is made of shape memory alloys e.g., Nitinol. Such implants don’t require any external force to regain their un-compressed structure. Due to shape-memory property, such implants start attaining their normal structure from an end once the capsule is moved to uncover the implant, starting from the end. The implant is situated over the guidewire shaft and in between the tip and the implant holder. The inner shaft extends longitudinally along the middle section from a proximal end of the distal section and further extends till a proximal end of the proximal section. The guidewire shaft extends from the proximal end of the proximal section till a distal end of the distal section.

The implant holder is a hub-like cylindrical structure fixed on the inner shaft and situated inside the capsule at a proximal end of the distal section. The implant holder has a plurality of pins on its peripheral surface. These pins are, optionally, at equal distance and angle from each other circumferentially. These pins are to get engaged with frame of the implant at the time of loading the implant and to get disengaged at the time of deployment. As mentioned above, in some cases and due to various reasons, the implant doesn’t get disengaged quickly and requires additional maneuvering to get the implant dislodged. This increases procedure time, and the positioning of the implant may also get affected.

The present disclosure utilizes a combination of design and material properties to address this issue of ensuring disengagement of the implant. The solution involves use of an elastic polymeric or metallic material whose thin strip is being placed between the peripheral surface of the implant holder and an engaging part of the frame of the implant. Preferably, the thin strip placement is around the pin of the implant holder. The thin strip is designed in such a way so that it acts as a cantilever or similar to a leaf spring whose ends are fixed. Also, in normal state, the distance between the peripheral surface of the implant holder and at least a portion of the thin strip is almost equal or lower than or higher than the height of the pin situated on the peripheral surface of the implant holder.

On loading the implant on the guidewire shaft and inside the capsule, the engaging part of the frame of the implant gets engaged with the pin of the implant holder and by design the portion of the thin strip is placed between the peripheral surface of the implant holder and the engaging part of the frame of the implant. In the implant loading procedure, the capsule is moved to compress and house the implant inside the hollow cylindrical structure of the capsule. In the process, the thin strip is also compressed, and potential energy is stored in the thin strip.

During deployment procedure, the capsule is moved to uncover the implant and the engaged part of the implant moves back to its original shape. In normal operation, this is sufficient to disengage the implant from the frame holder. However, the thin strip also tries to move back to its original position due to its elastic nature of the material used or by releasing the stored potential energy. This additional movement of the thin strip takes place simultaneously along with the disengagement of the engaged part of the implant and provides additional force to the engaged part for disengagement. In some cases, the engaged part of the frame of the implant doesn’t get disengaged on removal of the compressive force applied by the capsule. In such scenario, due to material property, the thin strip applies the additional force on the engaged part of the frame of the implant and the combined force of the additional force, due to potential energy release in the thin strip, and the shape memory property of the implant frame ensures disengagement of the implant from the catheter delivery system.

According to an embodiment of the present disclosure, the thin strip can be designed as a cantilever where one end of the thin strip is attached to a proximal end of the implant holder and the other end is free. In another embodiment, one end of the thin strip is attached to the distal end of the implant holder and the other end is free. In both the cases, the length of the thin strip is sufficiently long to cover or exceed at least half of the width of the pin. According to yet another embodiment, both the ends of the thin strip are fixed to the proximal end and the distal end of the implant holder respectively. However, the middle part of the thin strip is not connected to any surface. Also, the overall length of the thin strip is slightly higher than the distance between the points where the thin strip joins the implant holder. This gives the thin strip a bell-shaped configuration where a portion of the thin strip or the formed bell is near the pin and the height of a portion of the thin strip is almost equal or lower than or higher than the height of the pin.

According to yet another embodiment of the present disclosure, a combination of a plurality of the thin strips can be used which will act as combined cantilevers. In this embodiment, the plurality of thin strips in cantilever configuration are disposed parallel to each other or one thin strip superimposes another thin strip partially or completely. The length and location of the plurality of the thin strips may vary over peripheral surface of the implant holder. In one such embodiment, a second thin strip is situated between the proximal end of the implant holder and the distal end of the implant holder. One end of the second thin strip is attached to the peripheral surface of the implant holder whereas another end is free and, optionally, touches the superimposing or larger thin strip anywhere between the other free end of the thin strip and the joined end. The objective of the second thin strip is to provide additional force for detachment.

According to yet another embodiment of the present disclosure, a nested combination of the plurality of thin strips can be used i.e., the thin strip comprises at least a third thin strip that is attached to the thin strip and extends in space between the thin strip and the peripheral surface of the implant holder. During the procedure, the third thin strip provides additional force to the thin strip to cause detachment of the engaging part of the frame from the implant holder.

According to yet another embodiment of the present disclosure, the thin strip is designed as a cantilever where one end of the thin strip is attached to a proximal end of the implant holder and the other end is free. The thin strip has at least one step change curvature along the length of the cantilever. Due to the step-change curvatures, while being compressed by the capsule, the overall profile of the thin strip may increase in radial direction resulting in overall increase of profile of the capsule. A slot is created in the inner shaft to partially accommodate the thin strip, specifically, the middle portion of the thin strip, resulting in reduction of the overall profile of the thin strip in radial direction. The slot may be longitudinally extending or circumferentially extending. Also, the shape of the slot may be selected from, but not limited to, the rectangular, circular, helical, ovel, or irregularly shaped but accommodating the protruding parts of the thin strip or a combination thereof.

According to yet another embodiment of the present disclosure, at least one circumferential slot, is created on the inner shaft to increase flexibility of the catheter in area where the implant holder is located. The ends of the circumferential slots are not connected. The circumferential slots may partially overlap each other circumferentially or longitudinally.

According to yet another embodiment of the present disclosure, the thin strip covers the peripheral surface of the implant holder from the joined end till the pin or till the distal end of the implant holder with an eyelet on its peripheral surface to provide an access site that allows the thin strip to move without hinderance from the pin. In some embodiments, the size of the access site can extend along the length of the thin strip, and it can be of various shapes as well. Similarly, the thin strips can be of various sizes and shapes while conforming with the implant holder and the capsule in the compressed or normal state.

According to yet another embodiment of the present disclosure, the thin strip has at least one step change reduction in the thickness of the thin strip along the length of the cantilever or at least one step change curvature while maintaining the thickness of the thin strip along the length of the cantilever. The step change shape may be in shape of a stair or in curved shape or in a tapered shape. The step change shape allows to provide a push force to the engaging part of the implant frame, at the time of detachment.

According to yet another embodiment of the present disclosure, the implant may be, but not limited to, a stent, a valve, a mesh, a balloon, a patch, a drug-containing matrix, a shunt, or a combination thereof.

According to yet another embodiment of the present disclosure, the implant has at least one design element that can be engaged with the pin. Such a design element may be, but not limited to, a hook-shaped element, a ring-shaped element, a closed ring-shaped element, an open ring-shaped element, an irregular-close-shaped element, an irregular-open-shaped element, a U-shaped element, a V-shaped element, a W-shaped element, a M-shaped element, an end part of a stent, a valve or a shunt that can be put around the pin, part of a mesh, part of a frame, or a combination thereof.

According to yet another embodiment of the present disclosure, the pin can be of various sizes and shapes, specifically selected from, but not limited to, the rectangular, circular, D-shaped, ovel, hexagonal, pentagonal, octagonal, and triangular configurations.

The materials used for fabricating such cantilever is selected from, but not limited to, stainless steel, nitinol, polyamide, polypropylene, Acrylonitrile butadiene styrene and a combination thereof.

According to yet another embodiment, the implant holder comprises at least one radiopaque marker. The radiopaque marker is situated on the peripheral surface of the implant holder and its components including, but not limited to, the pin, the thin strip, the second thin strip, the third thin strip or a combination thereof.

According to yet another embodiment, a shape of the radiopaque marker present on the percutaneous catheter is selected from a circle, rectangular, square, oval, hexagonal, oblong, star-shaped, diamond-shaped, a circumferential ring, an irregular-shaped circumferential ring, an incomplete circumferential ring, an incomplete irregular circumferential ring or a combination thereof.

According to yet another embodiment, the implant is used in treating any abnormality or in any medical procedure related to heart, kidney, lever, brain, pancreas, lungs, digestive system, endovascular system, any tract, duct or any conduit in animal or human body. More specifically, the implant can be deployed in an artery, vein, heart valves, esophageal duct, bile duct, urinary tract, alimentary tract, tracheobronchial tree, cerebral aqueduct or genitourinary system of an animal or human body.

In addition, the present subject matter also envisages a method for fabricating the implant holder as explained above. For the manufacturing of the implant holder, the method requires or includes loading of a medically clean and approved workpiece in a designing instrument. According to one example of the present subject matter, the workpiece can be in shape of a hollow circular tube, or a solid cylinder, or a sheet. In some embodiments, the workpiece is prepared from a composition in powder form or prepared from a composition in liquid form. Then the required design of the implant holder is set-up or uploaded in the designing instrument, such as a computer-numerical controlled (CNC) machine for manufacturing. Subsequently, the required design is carved out of the workpiece to fabricate the implant holder. In one example, the fabrication technique used in the designing instrument is selected from laser fabrication, chemical-etching, mechanical machining, chemical machining, metal injection molding, vacuum casting, milling, photochemical-etching, electro-discharge machining, 3D-printing technique, additive manufacturing technique or a combination thereof. For instance, the implant holder is fabricated by slitting a metallic or polymeric hollow circular tube with a laser beam, the laser beam following a predefined cutting contour to produce the design of the implant holder. Alternatively, the implant holder is be manufactured using 3D printing technique or additive manufacturing. Once the implant holder has been manufactured, the undesired material is removed from the surface of the implant holder for finishing. The cleaned and finished implant holder can then be polished or coated with an appropriate coating. For example, it can be coated with an anti-reactive agent which prevents it from reacting with the atmosphere where either the implant is stored or deployed. Additionally, or alternatively, the implant holder can be covered with a medicinal substance or radiopaque substance, depending on the purpose, mode, and location of deployment of the implant holder.

3D printing technique can be selected from but not limited to Stereolithography (SLA), Digital light processing (DLP), Fused deposition modelling (FDM), Selective laser sintering (SLS), Selective laser melting (SLM), Electronic beam melting (EBM), Laminated object manufacturing (LOM), Polyjet technology or a combination of thereof.

By combining different materials and design variations explained above, a variety of configurations can be obtained with varying structure-property relationships.

Now, referring to the figures, wherein the elements are labelled with like numerals throughout the several Figures. Further, reference is made to the accompanying drawings which form a part hereof, and in which is shown by way of illustration a specific embodiment. It is to be understood that other embodiments may be utilized, and structural changes may be made without departing from the scope of the present invention.

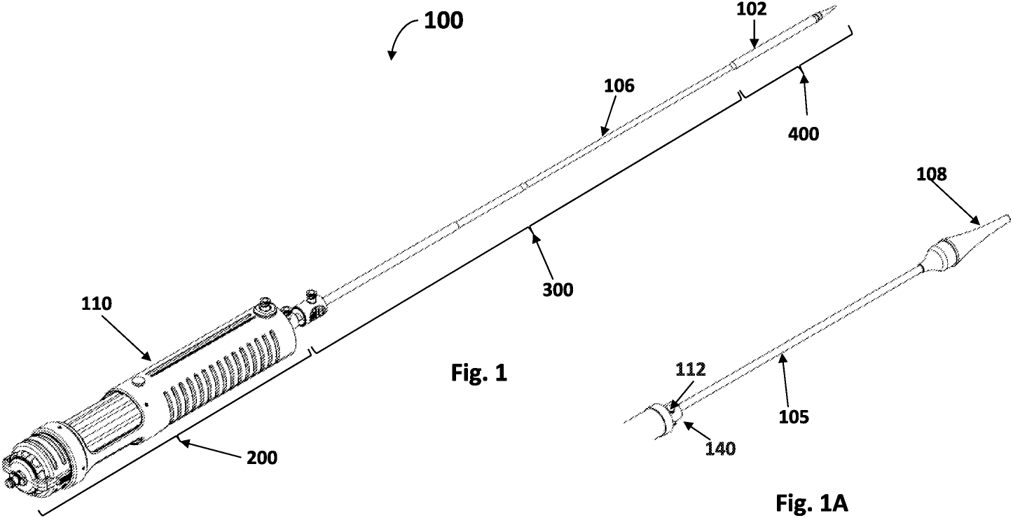

FIG. 1 and FIG. 1A represent, according to an embodiment of the present disclosure, a typical catheter delivery system. The catheter delivery system (100) comprises a distal section (400), a middle section (300), and a proximal section (200). The proximal section (200) remains outside the human body and comprises a handle (110) to control the movements at the distal section (400) of the catheter. The distal section comprises a tip (108), a guidewire shaft (105), an implant holder (140) and a capsule (102). The middle section (300) is connected proximally with the handle housing and distally it connects to the distal section (400). The middle section (300) mainly comprises a catheter shaft (106) that connects to the capsule (102) in the distal section (400).

The capsule (102) provides an inside space where the implant is loaded in compressed form and the capsule (102) helps in retaining the implant in compressed form. The implant is situated over the guidewire shaft (105) and in between the tip (108) and the implant holder (140). The implant holder (140) is fixed on the inner shaft (104) and situated inside the capsule (102) at a proximal end of the distal section (400). In certain example embodiments, the implant holder (140) has a plurality of pins (e.g., as shown at 112, 202, 302, 402, 502, 602, 702, or 802) to get engaged with the frame of the implant.

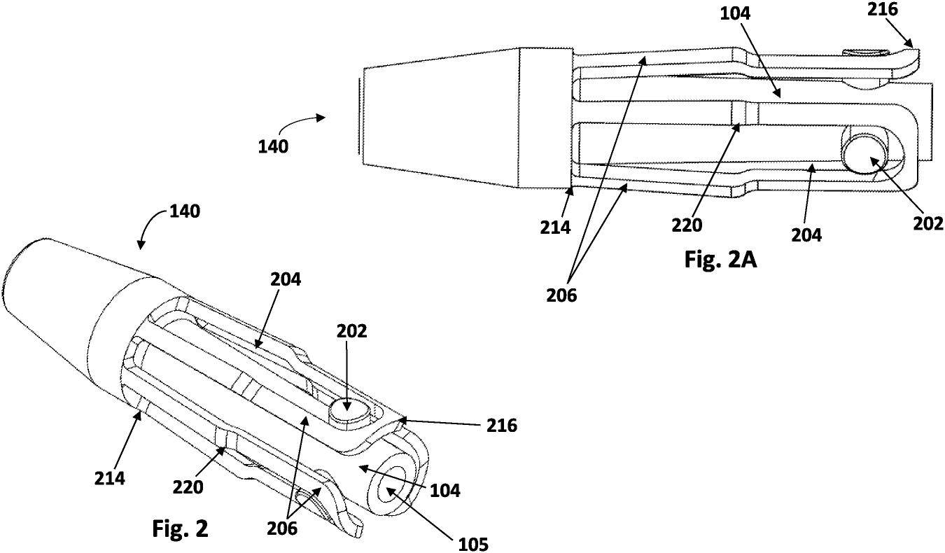

Referring to FIG. 2 and FIG. 2A, according to an embodiment of the present disclosure, a thin strip (206), which is designed as a cantilever, has one end (214) attached to a proximal end of the implant holder and the other end (216) is free. The thin strip (206) covers, partially, the peripheral surface of the implant holder from the one end till the pin (202) or till the distal end of the implant holder while, optionally, having an elongated slot (204) on its peripheral surface to provide a space to the pin (202) while the thin strip (206) moves in radial direction. Further, optionally, the thin strip (206) has at least one step change (220) in the thin strip along its length.

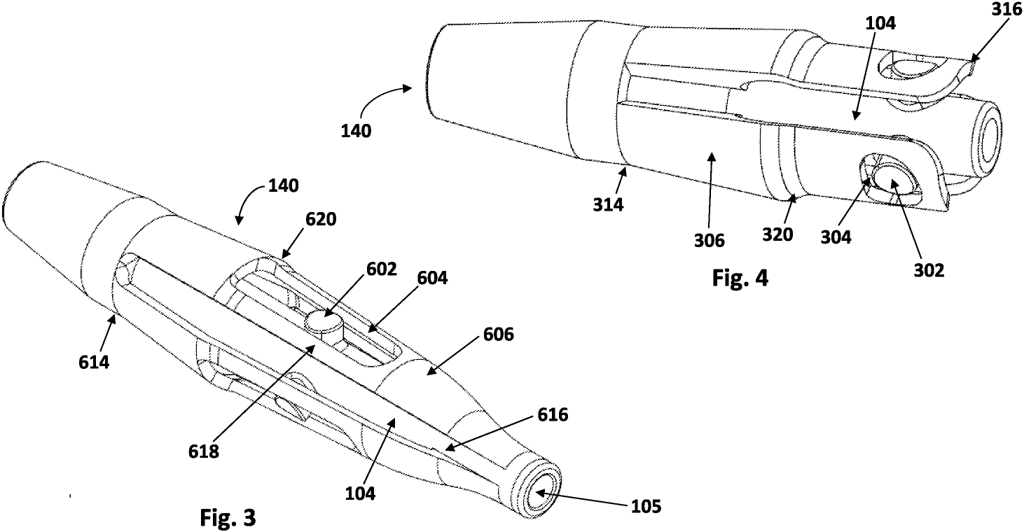

Referring to FIG. 3, according to another embodiment of the present disclosure, both the ends, the one end (614) and the other end (616) of the thin strip (606) are fixed to the proximal end and the distal end of the implant holder (140) respectively. However, the middle portion (618) of the thin strip (606) is not attached to any surface. This gives the thin strip (606) a bell-shaped configuration where a part of the middle portion (618) of the thin strip or the formed bell is near the pin (602). Due to the formed bell, fixed ends, and elastic property of the thin strip; the thin strip (606) behaves similar to a leaf spring. In addition, the thin strip (606) has an elongated slot (604) on its peripheral surface to provide a space to the pin (602) while the thin strip (606) moves in radial direction. Further, optionally, the thin strip (606) has at least one step change (620) in the thin strip along its length.

Referring to FIG. 4, according to yet another embodiment of the present disclosure, a thin strip (306), which is designed as a cantilever, has a one end (314) attached to a proximal end of the implant holder and the other end (316) is free. The thin strip (306) covers, partially, the peripheral surface of the implant holder from the one end till the pin (302) or till the distal end of the implant holder while, optionally, having an eyelet (304) on its peripheral surface to provide a space to the pin (302) while the thin strip (306) moves in radial direction. Further, optionally, the thin strip (306) has at least one step change (320) in the thin strip along its length.

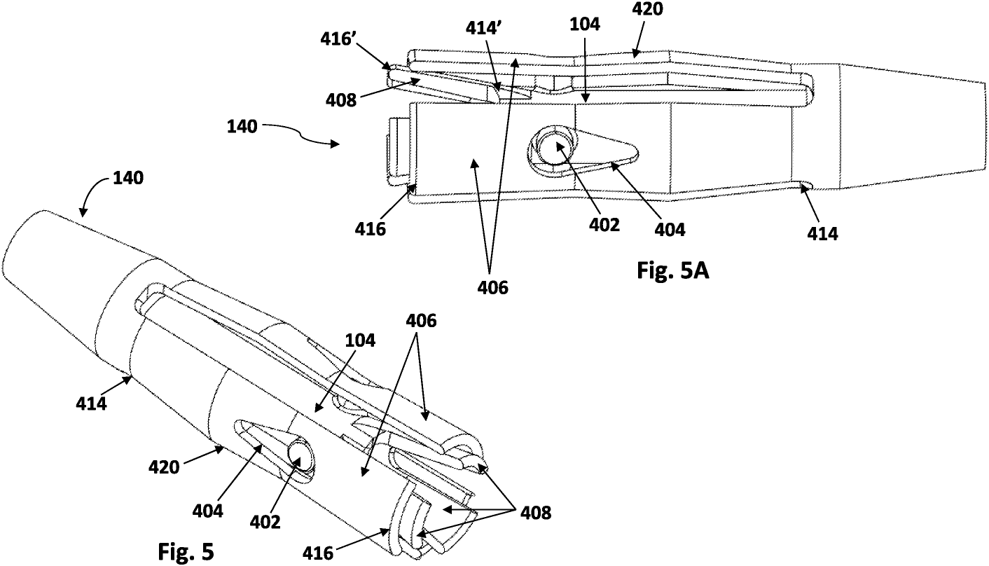

Referring to FIGS. 5 and 5A, according to yet another embodiment of the present disclosure, thin strip (406), which is designed as a cantilever, has one end (414) attached to a proximal end of the implant holder and the other end (416) is free. The thin strip (406) superimposes a second thin strip (408) partially or completely. Similar to the thin strip (406), the one end (414′) of the second thin strip (408) is connected to the peripheral surface of the implant holder and the other end (416′) is free. The thin strip (406) covers, partially, the peripheral surface of the implant holder from the one end till the pin (402) or till the distal end of the implant holder while, optionally, having an eyelet (404) on its peripheral surface to provide a space to the pin (402) while the thin strip (406) moves in radial direction. Further, optionally, the thin strip (406) has at least one step change (420) in the thin strip along its length.

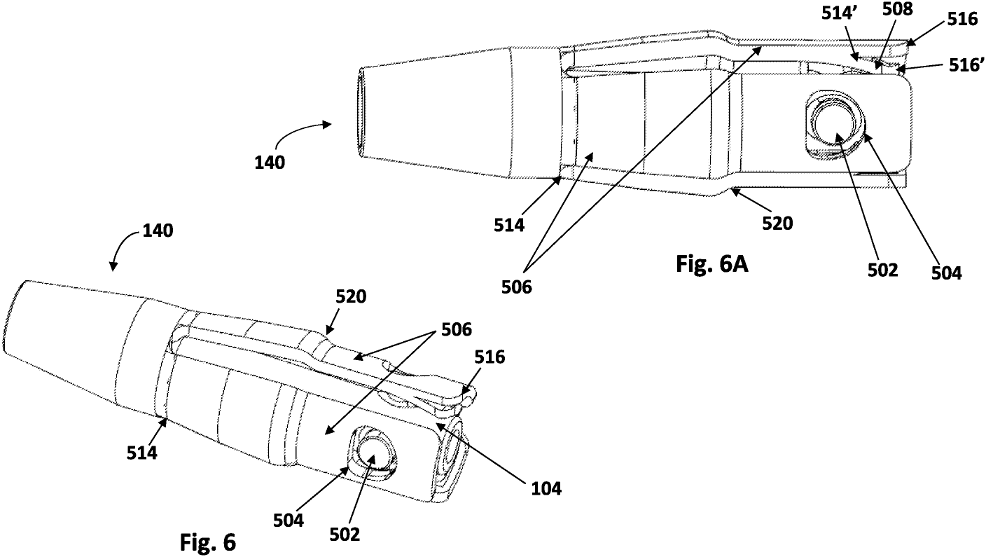

Referring to FIGS. 6 and 6A, according to yet another embodiment of the present disclosure, thin strip (506), which is designed as a cantilever, has one end (514) attached to a proximal end of the implant holder and the other end (516) is free. A third thin strip (508) is attached to the thin strip (506) in a nested configuration. In nested configuration, the one end (514′) of the third thin strip (508) is connected to the thin strip and the other end (516′) extends in space between this strip and the peripheral surface of the implant holder (140). The thin strip (506) covers, partially, the peripheral surface of the implant holder from the one end till the pin (502) or till the distal end of the implant holder while, optionally, having an eyelet (504) on its peripheral surface to provide a space to the pin (502) while the thin strip (506) moves in radial direction. Further, optionally, the thin strip (506) has at least one step change (520) in the thin strip along its length.

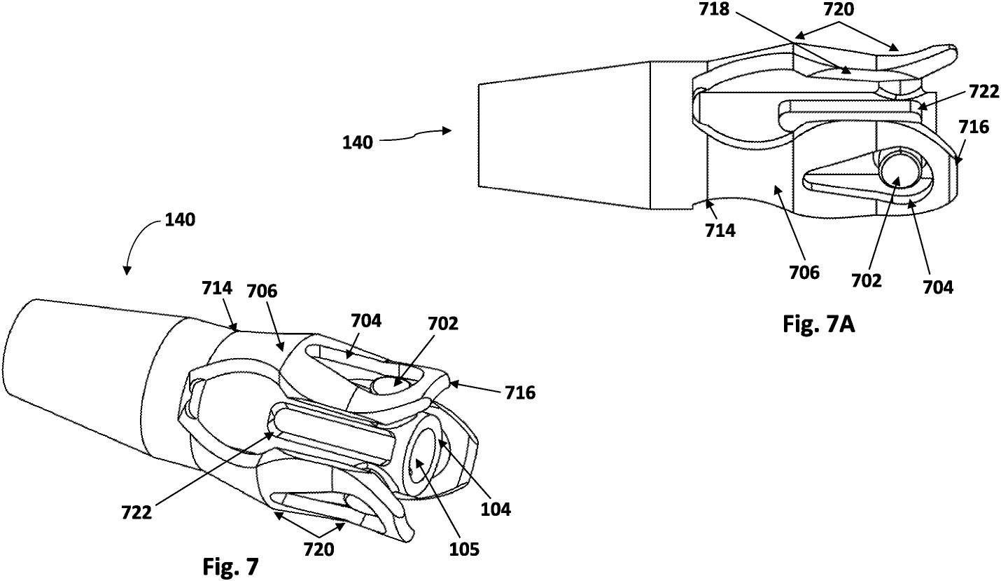

Referring to FIGS. 7 and 7A, according to yet another embodiment of the present disclosure, the one end (714) of the thin strip (706) is attached to proximal side of the implant holder (140) and the other end (716) is free to move in radial direction based on compressive force applied or removed by the capsule. The thin strip (706) covers, partially, the peripheral surface of the implant holder from the one end till the pin (702) or till the distal end of the implant holder while having an eyelet (704) on its peripheral surface to provide a space to the pin (702) while the thin strip (706) comes down in radial direction. Further, the thin strip (706) has at least one step change (720) in the thin strip along its length. Due to the step-changes (720), the overall profile of the thin strip (706) increases in radial direction. Hence, a slot (722) is created in the inner shaft (104) to partially accommodate the middle portion (718) of the thin strip, while being compressed by the capsule, resulting in reduction of the overall profile of the thin strip (706) in radial direction.

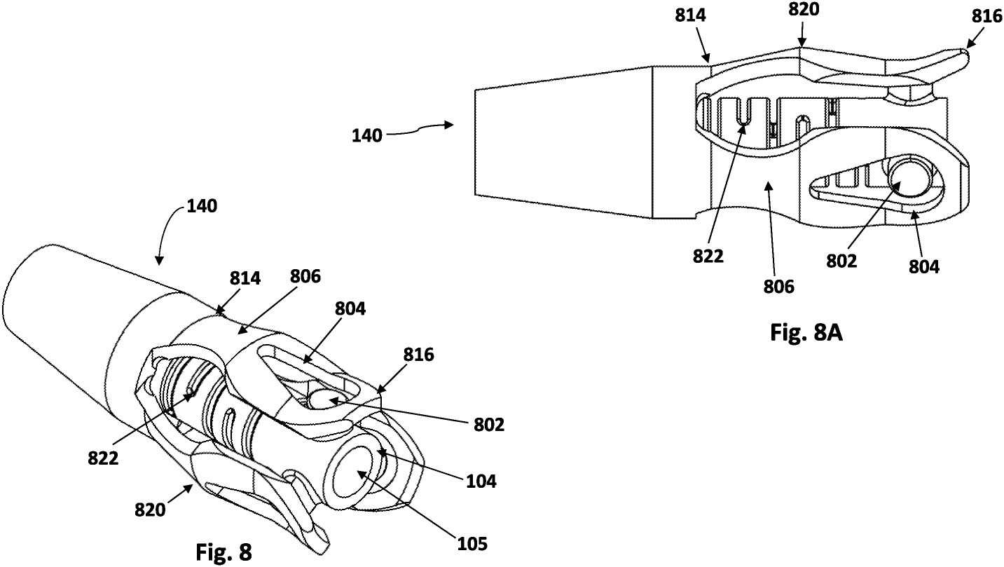

Referring to FIGS. 8 and 8A, according to yet another embodiment of the present disclosure, the one end (814) of the thin strip (806) is attached to proximal side of the implant holder (140) and the other end (816) is free to move in radial direction based on compressive force applied or removed by the capsule. The thin strip (806) covers, partially, the peripheral surface of the implant holder from the one end till the pin (802) or till the distal end of the implant holder while having an eyelet (804) on its peripheral surface to provide a space to the pin (802) while the thin strip (806) comes down in radial direction. The thin strip (806) has at least one step change (820) in the thin strip along its length. Further, at least one circumferential slot (822) is created on the inner shaft (104) to increase flexibility of the catheter in area where the implant holder (140) is located.

In the present description, for purpose of explanation, specific details are set forth in order to provide an understanding of the present disclosure. It will be apparent, however, to one skilled in the art that other embodiments may be practiced apart from the specific details described herein. One skilled in the art will recognize that specific details described herein may be incorporated into other embodiments, structures and devices shown in the figures are illustrative according to the exemplary embodiments. In other instances, detailed descriptions of well-known methods, devices, techniques, etc. are omitted so as not to obscure the description with unnecessary detail.

### List of Reference Numerals

Capsule - 102
Inner shaft - 104
Guidewire shaft - 105
Catheter shaft - 106
Tip - 108
Handle - 110
Elongated slot - 204, 604
Implant holder - 140
Pin - 112, 202, 302, 402, 502, 602, 702, 802
Thin strip - 206, 306, 406, 506, 606, 706, 806
Eyelet - 304, 404, 504, 704, 804
One end - 214, 314, 414, 414′, 514, 514′, 614, 714, 814
Other end - 216, 316, 416, 416′, 516, 516′, 616, 716, 816,
Step-change - 220, 320, 420, 520, 620, 720, 820
Proximal section - 200
Middle section - 300
Distal section - 400
Second thin strip - 408
Third thin strip - 508
Middle portion - 618, 718
Slot - 722
Circumferential slot - 822

What is claimed is:

1. An implant detaching mechanism configured to detach an implant from an implant holder, comprising:
a catheter having an inner shaft, a catheter shaft, a capsule, and a handle;
the implant holder is attached to the inner shaft, the implant holder having at least one pin on its peripheral surface that is configured to engage an implant to the implant holder;
wherein the catheter shaft is movable in the longitudinal direction and a capsule is attached to the distal end of the catheter shaft, wherein the capsule is configured to cover and uncover, upon movement of the catheter shaft, the implant holder and, at least partially, the implant when the implant is engaged to the implant holder ; and
a thin strip, elastic in nature, that is attached to the implant holder and located between the peripheral surface of the implant holder and the implant when the implant is engaged to the implant holder;
wherein, on the capsule being moved to cover the implant holder, a force is applied on the thin strip and the implant when the implant is engaged to the implant holder;
wherein, on the capsule being moved to uncover the implant holder, release of potential energy stored in the thin strip is configured to cause detachment of the implant, when the implant is engaged to the implant holder, from the pin.

2. The implant detaching mechanism as claimed in claim 1, wherein the thin strip in the implant holder is attached towards proximal side of the implant holder at a first end of the thin strip and a second, opposing, end of the thin strip is free.

3. The implant detaching mechanism as claimed in claim 1, wherein the thin strip in the implant holder is attached towards a proximal side of the implant holder at a first end of the thin strip and a second end of the thin strip is attached towards distal side of the implant holder, wherein a length of the thin strip is higher than a distance between the attached ends to the thin strip into a curved configuration.

4. The implant detaching mechanism as claimed in claim 1, wherein the thin strip in the implant holder has at least a length long enough: 1) to reach the closest peripheral surface of the pin, 2) to reach the center point of the pin, 3) to reach the farthest peripheral surface of the pin, or 4) to surpass the pin.

5. The implant detaching mechanism as claimed in claim 1, wherein the thin strip in the implant holder has an eyelet or an elongated slot on its peripheral surface that is configured to accommodate an overlapping portion of the pin on compression due to the movement of the capsule.

6. The implant detaching mechanism as claimed in claim 1, wherein the implant holder has a plurality of pins attached to the implant holder at equal distance in circumferential direction.

7. The implant detaching mechanism as claimed in claim 1, wherein the implant holder has a plurality of pins attached to the implant holder at unequal distance in circumferential direction.

8. The implant detaching mechanism as claimed in claim 1, wherein the thin strip in the implant holder has at least a step-change reduction in the thickness along its length or along its circumference.

9. The implant detaching mechanism as claimed in claim 1, wherein the thin strip in the implant holder has at least a step-change in the curvature along its length or along its circumference.

10. The implant detaching mechanism as claimed in claim 1, wherein the thin strip includes at least one second thin strip attached to the thin strip.

11. The implant detaching mechanism as claimed in claim 1, wherein the implant holder has a plurality of thin strips attached to the implant holder, wherein the plurality of thin strips are disposed parallel to each other.

12. The implant detaching mechanism as claimed in claim 1, wherein the implant holder is attached to the inner shaft, wherein the inner shaft has at least one slot extending longitudinally or circumferentially.

13. The implant detaching mechanism as claimed in claim 12, wherein the implant holder is attached to the inner shaft wherein shape of the slot is rectangular, circular, helical, oval, or irregularly shaped configuration.

14. The implant detaching mechanism as claimed in claim 12, wherein the implant holder is attached to the inner shaft wherein at least one edge of the thin strip is accommodated in the slot.

15. The implant detaching mechanism of claim 1, wherein the implant is selected from a stent, a valve, a mesh, a balloon, a patch, a drug-containing matrix, a shunt, a vena cava filter, a vascular graft, a stent graft, and/or a combination thereof.

16. The implant detaching mechanism of claim 1, wherein the pin on the implant holder has a cross-sectional shape selected from rectangular, circular, D-shaped, oval, hexagonal, pentagonal, octagonal, triangular configuration, and/or a combination thereof.

17. The implant detaching mechanism of claim 1, wherein the implant holder is made of a biocompatible material selected from a group of polymers, metals, alloys, non-metals, biodegradable materials, bioresorbable materials, and/or a combination of thereof.

18. The implant detaching mechanism of claim 1, wherein the implant holder is made of a biocompatible material selected from stainless steel, nitinol, cobalt-chromium, polyamide, polypropylene, Acrylonitrile butadiene styrene, and/or a combination thereof.

19. The implant detaching mechanism of claim 1, wherein the implant holder has at least one radiopaque marker on its peripheral surface.

20. A method of manufacturing the implant holder as claimed in claim 1, the method comprising the step of:
setting-up a design of the implant holder in a designing instrument;
carving the design on a work piece to fabricate the implant holder;
finishing the implant holder by removing material from a surface of the implant holder and polishing the implant holder; and
fixing the implant holder on the inner shaft.

21. The method as claimed in claim 20, wherein the work piece is one of a hollow circular tube, or a solid cylinder, or a sheet, or prepared from a composition in powder form or prepared from a composition in liquid form.

22. The method as claimed in claim 20, wherein the step of carving is selected from at least one of laser fabrication, chemical-etching, mechanical machining, chemical machining, metal injection molding, vacuum casting, milling, photochemical-etching, electro-discharge machining, 3D-printing technique, additive manufacturing technique or a combination thereof.
